(19) United States
(12) Patent Application Publication
(10) Pub. No.: US 2003/0105293 A1
(43) Pub. Date: Jun. 5, 2003

(54) HUMAN CIRCULATING CYTOKINE CC-1

(76) Inventors: WOLF-GEORG FORSSMANN, HANNOVER (DE); PETER SCHULZ-KNAPPE, HANNOVER (DE); MARKUS MEYER, HANNOVER (DE); HANS-JURGEN MAGERT, HANNOVER (DE)

Correspondence Address: JACOBSON PRICE HOLMAN & STERN, 400 SEVENTH STREET NW, WASHINGTON, DC, 20004

(21) Appl. No.: 08/666,340

(22) PCT Filed: Dec. 22, 1994
(86) PCT No.: PCT/EP94/04282

(30) Foreign Application Priority Data
Dec. 24, 1993 (DE) 43 44 397.4
Aug. 3, 1994 (DE) 44 27 395.9

(51) Int. Cl. C07H 21/04; C07K 1/00; C07K 14/00; C07K 17/00; C07K 16/00; C12P 21/08

(52) U.S. Cl. 530/351; 536/23.5; 530/387.9; 536/24.3

(57) ABSTRACT

Cytokine CC-1 having the amino acid sequence according to SEQ ID No. 6 and its biologically active fragments and/or derivatives, in particular amidized, acetylized, phosphorylized and/or glycosylized derivatives.

The present invention pertains to a polypeptide from the class of cytokines, cytokine CC-1, as well as its biologically active fragments and/or derivatives, a polynucleotide coding for said cytokine CC-1 or its biologically active fragments, in particular a cDNA, a medicament containing the peptide according to the invention, a diagnostic agent, the use of cytokine CC-1 for second medical indications, and a nucleic acid probe hybridizing to a polynucleotide coding for cytokine CC-1 or one of its fragments.

Surprisingly, it has been shown that a cytokine CC-1 can be isolated from human hemofiltrate. This cytokine has the amino acid sequence given in SEQ ID No. 6.

Fragments of cytokine CC-1 also have biological activity. The fragments can be obtained by methods known to one skilled in the art, for example, by digestion with peptidases, especially endoproteases. Fragmentation of the peptide according to the invention by means of chemical reagents cleaving peptide bonds, especially cyanogen bromide, also yields biologically active fragments.

The peptide according to the invention can be obtained by an isolation procedure departing from human hemofiltrate.

The human hemofiltrate is optionally diluted with water and acidified. The pH value is preferably from 1.5 to 3.5, in particular from 2.5 to 3.0. Then, the hemofiltrate is treated with a cation exchanger, for example, a support material modified with sulfonic acid groups (Fractogel medium SO₃⁻ of Merck). The peptides bound to to the cation exchanger are eluted with relatively highly concentrated saline in an acid pH range corresponding to that above given. The ionic strength of the eluent approximately corresponds to a 0.7 to 1.3 molar sodium chloride solution.

The eluate collected is spiked with a peptide-precipitating reagent, e.g., ammonium sulfate. The precipitation of the peptides is preferably performed at lower temperatures, in particular in the range of from 4 to 10° C. The precipitate thus obtained is freed from the supernatant, taken up in water, and then a peptide-precipitaing lower alcohol, such as isopropanol, is added. This is followed by another cation exchange chromatography. This chromatography is preferably a gradient elution chromatography with a buffer from low ionic strength to one of higher ionic strength, corresponding an ionic strength of about from 0.7 to 1.3 M NaCl.

The biologically active fragments are pooled and further purified by preparative reversed phase chromatography on support materials modified with C4. Further chromatographic purification steps may follow, if required.

The material obtained by chromatographical purification was subjected to a structure determination. Sequence analysis was performed via an Edman degradation of the peptide and the cleavage products by means of an ABI 473 A sequencer.

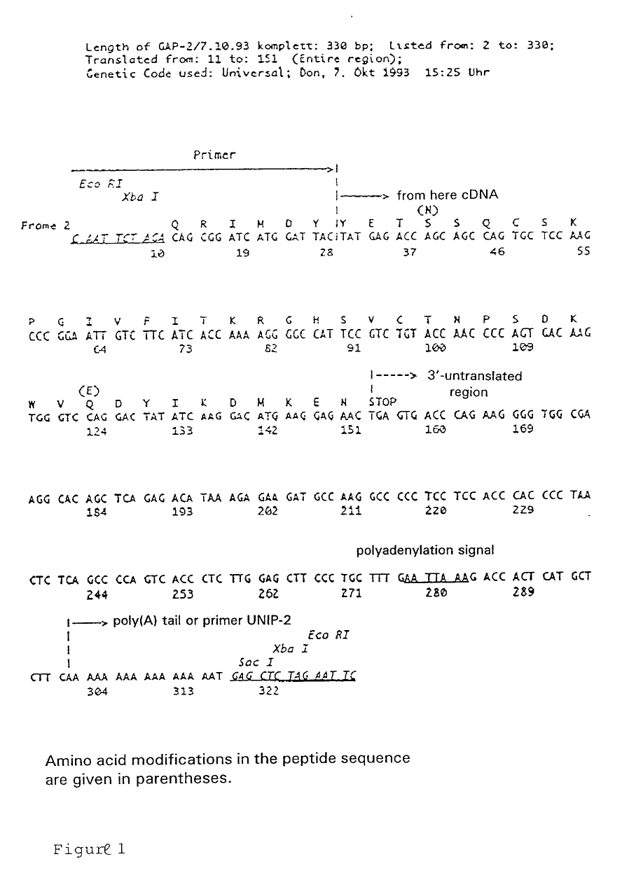

From the peptide sequence according to the invention, a polynucleotide can be derived coding for the cytokine CC-1 (FIG. 1) having the C-terminal fragment according to SEQ ID No. 8 and the nucleic acid sequence SEQ ID No. 9 linked thereto.

In particular, said polynucleotide is a cDNA which may serve as both the starting point of a genetic engineering preparation of the cytokine CC-1 and as an analytical tool for the detection of the presence of DNA or mRNA coding for the protein.

Appropriate derivatives may be employed as hybridization probes. For instance, the cDNA coding for a fragment of the peptide according to the invention has the sequence according to SEQ ID No. 7.

In addition to a genetic engineering preparation, a stepwise total synthesis on usual solid phases in terms of Merrifield synthesis is also possible. The strategy of synthesis and the construction of the peptide with the correspondingly protected amino acids are known to one skilled in the art.

The peptide according to the invention may be used as a medicament. Its biological activity is that of a cytokine. Therefore, it may be employed as a medicament in the indications given in claim 7. The peptide according to the invention may be administered, as is common with peptides, parenterally, intravenously or intramuscularly, or intranasally or bucally. The amount of peptide to be administered is between 10 and 3000 μg per dosage unit.

The diagnostic agent according to the invention contains polyclonal or monoclonal antibodies against the peptide according to the invention, optionally in fluorescence-labeled or radioactively labeled form, to be employed in per se known ELISA or RIA assays.

The invention will be described in more detail by means of the following examples.

### EXAMPLE 1

Five hundred 1 of human hemofiltrate were diluted to 2000 l with water, and the pH adjusted to 2.7 with concentrated HCl. After charging on an Amicon Vantage column (filling material, Merck Fractogel medium SO₃⁻), the bound peptides were eluted with 1 M NaCl, pH 3.0.

The eluate (7 l) was spiked with ammonium sulfate, and the peptides precipitated overnight at 4° C. The peptide precipitate was filtered through a Büchner funnel.

The precipitate obtained was dissolved in 2 l of water, and 4.5 parts of isopropanol were added. The precipitated peptides were again filtered through a Büchner funnel.

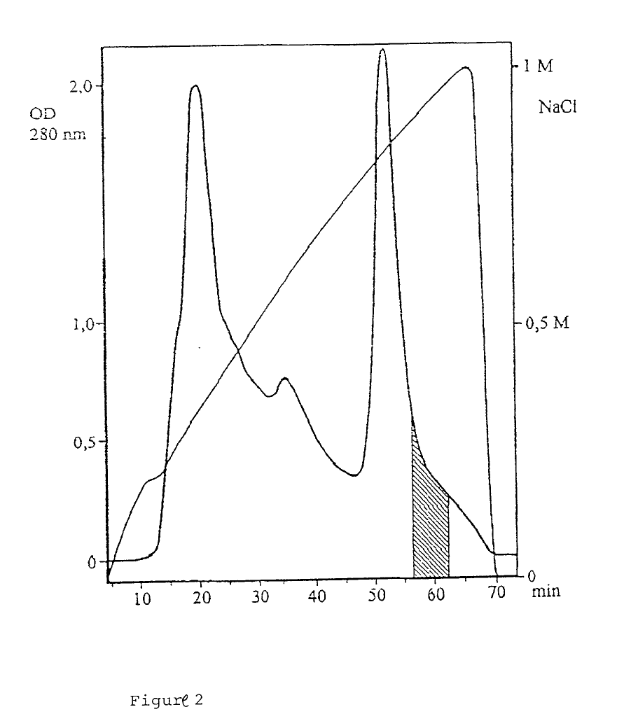

The precipitate after the isopropanol precipitation was dissolved in 4 l of water, and a pH of 3.0 was adjusted with HCl. After charging on a cation exchanger (column: Amicon Vantage), the column was eluted and the fractions collected (chromatograph see FIG. 2).

Chromatographic Conditions

Buffer A: 10 mM sodium dihydrogenphosphate, pH 3.0

Buffer B: buffer A with 1 M NaCl

Gradient: from 0 to 100% of B in 60 min

Flow: 40 ml/min

Detection: 280 nm

Chromatographic equipment: Biopilot (Pharmacia)

Fractions: 2 min each from the beginning of the gradient

Fractions 31 to 34 were pooled for further treatment.

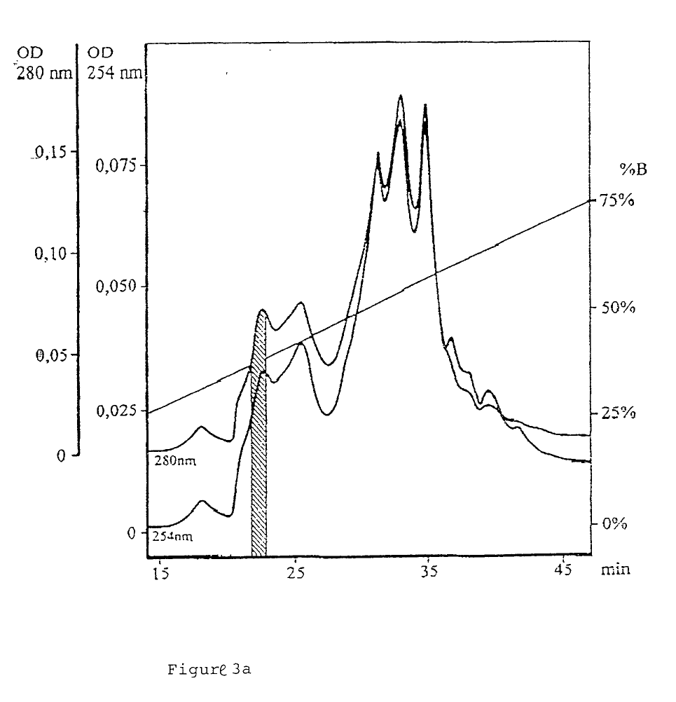

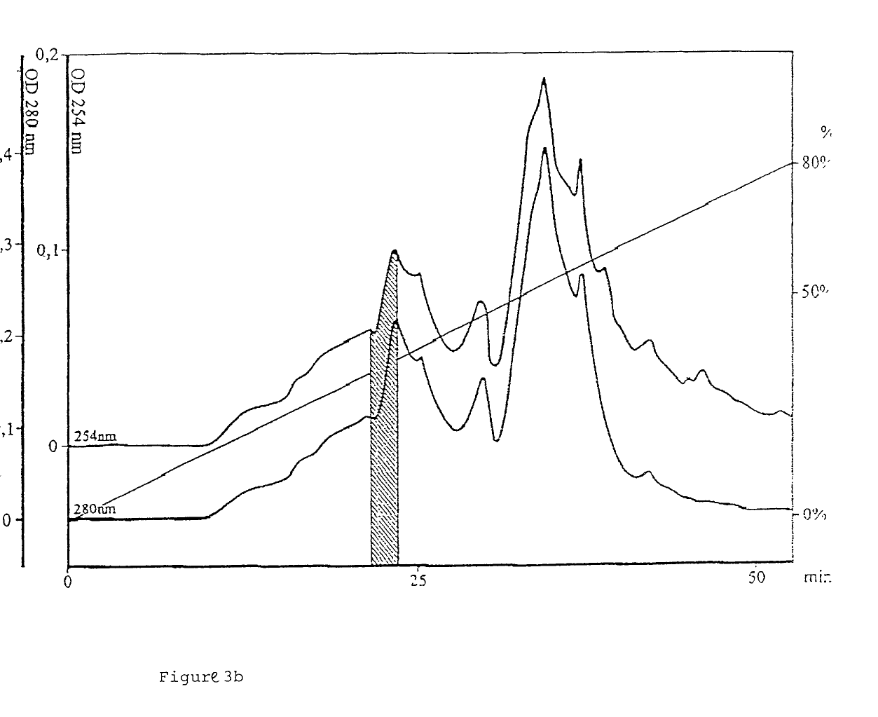

The pooled fractions 31 to 34 were successively separated in two chromatographic runs via a preparative reversed-phase column (chromatograph see FIGS. 3a and b).

Chromatographic Conditions

Column: 3 cm×12.5 cm steel column

Filling material: Parcosil RP-C4 25-45, 300 A

Buffer A: 0.01 N HCl

Buffer B: buffer A with 30% of methanol and 50% of isopropanol

Gradient: from 0 to 100% of B in 60 min

Flow: 15 ml/min

Detection: 280/254 nm

Chromatographic equipment: BioCAD (Perseptive)

Fractions: 1 min each from the beginning of the gradient

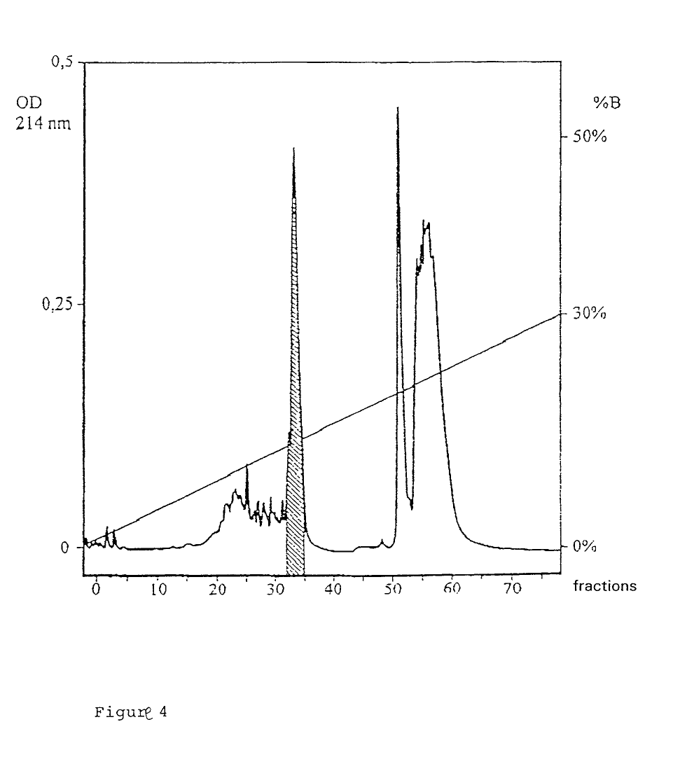

Fractions 22 and 23 from the first preparative run and fraction 24 of the second run were pooled and the solvent stripped off by a rotary evaporator. Then, the fractions were separated via a semi-preparative RP-C4 column (chromatograph see FIG. 4).

Chromatographic Conditions

Column: 1 cm×12.5 cm steel column

Filling material: Parcosil RP-C4 5 , 300 A

Buffer A: 0.1% of TFA

Buffer B: buffer A with 80% of acetonitrile

Gradient: from 0 to 30% of B in 60 min

Flow: 2 ml/min

Detection: 214 nm

Chromatographic equipment: Kontron 322

Fractions: 1 min each from the beginning of the gradient

Fractions 33 and 34 contain the substance, purified to more than 95%, the structure of which was elucidated in the following:

### EXAMPLE 2

Sequence Determination

Edman degradation of the peptide as well as the cleaving products was performed via an ABI 473 A sequencer after charging onto a Polybrene membrane in quantities of between 100 and 400 pmol using the standard program.

Determination of Cysteines

¹⁴C carboxymethylation and subsequent purification via an analytical Vydac C18 RP column (4.6 mm×25 cm). Detection of the carboxymethylated fraction in the radioactivity monitor.

Subsequently, Lys-C cleavage of 50% of the carboxymethylated peak with the endopeptidase, Lys-C. The cleavage was performed at 37° C. for 3 hours in the buffers given by the manufacturer (Boehringer, Mannheim) at a ratio of enzyme to peptide of 1:25. The cleavage products were separated by RP chromatography via an analytical Vydac C18 column. Pooling of the individual peaks and sequencing for a complete determination of the sequence.

Determination of the C-Terminus

The cleavage of the residual 50% of the carboxymethylated peptide is performed by means of chymotrypsin in the buffers given by the manufacturer (Boehringer, Mannheim) at a ratio of enzyme to peptide of 1:25, the subsequent purification is performed via an analytical Vydac C18 RP column (4.6 mm×25 cm). The individual peaks are pooled and analyzed for a complete determination of the sequence.

Determination of Molar Mass

The determination of the molar mass of the total peptide is performed by a Sciex API III, and of the fragments following Lys-C and chymotrypsin cleavage.

Sequencing and determination of the molar mass yield the sequence given above having a molar mass of 8689 Dalton.

A data bank comparison was performed on Swiss-Prot and EMBL-Peptid and Nukleinsauredatenbank. A sequence homology was established to various members of the superfamily of intercrines with a maximum homology to macrophage inflammatory protein MIP I alpha and MIP I beta.

### EXAMPLE 3

Determination of cDNA

Cloning and Characterization of a Partial Human Cytokine CC-1 cDNA Fragment

From human adrenal tissue, whole RNA was prepared by means of an automated nucleic acid extractor (ABI,340).

The mRNA from 5 μg of this RNA was transcribed into cDNA first strand using MMLV RTase (Gibco-BRL) and a synthetic oligo(dT) primer (UNIP-2, CCTGAATTCTAGAGCTCA(T)₁₇). At the same time, two “degenerated” PCR primer pairs were synthesized departing from the known peptide sequence which contained all coding possibilities for the corresponding amino acid sequences (see separate sheet “CC-1 amino acid sequence and PCR primers derived therefrom”). The first primer pair (CC-1-2/1, CC-1-2/2) was rather N-terminally localized with respect to the amino acid sequence whereas the position of the second primer pair (CC-1-2/3, CC-1-2/4) was shifted to the C-terminus. This was intended to enable an amplification in two stages (preamplification, reamplification) in order to increase the specifity of the reaction. The following reactions were performed:

1. In two different reactions, 1/15 each of the cDNA product was subjected to 40 PCR cycles with the primer combinations CC-1-2/1/UNIP-2 and CC-1-2/2/UNIP-2, respectively (pre-amplification, 2 batches). One cycle consisted of:

| | | | |
|---|---|---|---|
| 95° C. | 30 | s | denaturing |
| 48° C. | 30 | s | primer hybridization |
| 72° C. | 3 | min | extension |

2. Then, 1/30 each of the two products were reamplified in 20 cycles with the primer combinations CC-1-2/3/UNIP-2 and CC-1-2/4/UNIP-2, respectively (reamplification, 4 batches):

| | | | |
|---|---|---|---|
| 95° C. | 30 | s | denaturing |
| 42° C. | 30 | s | primer hybridization |
| 72° C. | 2 | min | extension |

By reamplification with CC-1-2/4/UNIP-2, a homogeneous PCR product could be obtained (see “agarose gel electrophoresis of the PCR fragments”). The PCR product was freed from unreacted primers by Centrikon C-100 (Amicon) centrifugation, restricted together with 50 ng of pBluescript Eco-RI (the PCR primers are given ECO-RI restriction sites for easier cloning), and subsequently ligated. The ligation products were propagated in E. coli XL-1 Blue, the plasmid DNA of white colonies prepared with Qiagen columns (Diagen) and sequenced by means of a fluorescence sequencer. The cloned cDNA can now be employed as a highly specific hybridization probe for screening a cDNA or gene library. In addition, specific primers for a direct amplification of the residual cDNA from the whole DNA of a human cDNA library can be derived from this sequence.

GAP-2 amino acid sequence and PCR primers derived therefrom

Primers

| | | |
|---|---|---|
| CC-1-2/4 | SEQ ID No. 1, +++! | 48 variations |
| CC-1-2/1 | SEQ ID No. 2, | 768 variations |
| CC-1-2/3 | SEQ ID No. 3, | 24 variations |

(coding for fragment SEQ ID No. 4 GAP-2 AA seq.)

| | | |
|---|---|---|
| CC-1-2/2 | SEQ ID No. 5, | 384 variations |
| CC-1-2/3 | SEQ ID No. 6, | 24 variations |

1. Cytokine CC-1 having the amino acid sequence according to SEQ ID No. 6 and its biologically active amidized, acetylized, phosphorylized and/or glycosylized derivatives.

2. A polynucleotide coding for cytokine CC-1 of claim 1.

3. The polynucleotide according to claim 2, characterized by being a cDNA fragment according to SEQ ID No. 7.

4. A process for the preparation of a cytokine CC-1 of claim 1 by the extraction of hemofiltrate, cation-exchange extraction, followed by elution of the adsorbed substances;
ammonium sulfate precipitation of the peptides and proteins present in the eluate;
taking up the precipitate in aqueous solution and once more precipitation with a lower alcohol and cation-exchange chromatography, and reversed-phase chromatography.

5. A medicament containing cytokine CC-1 of claim 1 as the active ingredient.

6. A diagnostic agent containing polyclonal or monoclonal antibodies against cytokine CC-1 of claim 1, or the nucleic acid or mRNA coding for said cytokine.

7. Use of cytokine CC-1 of claim 1 for the preparation of a medicament for the treatment of disturbances of cell migration, diseases of the immune system, tumors, and dysfunctions of regulatory growth functions.

8. Nucleic acid probes hybridizing to a polynucleotide of claim 2.


SEQUENCE LISTING (11 sequences)

SEQ ID NO: 1 - DNA (other nucleic acid), 32 bp; single-stranded, linear
  description: /desc = “PCR primer”
gcccggaattctagacarcgnathatggayta

SEQ ID NO: 2 - DNA (other nucleic acid), 32 bp; single-stranded, linear
  description: /desc = “PCR primer”
cccgaattctagaartayccnathccncgnca

SEQ ID NO: 3 - DNA (other nucleic acid), 32 bp; single-stranded, linear
  description: /desc = “PCR primer”
gcccggaattctagacaragrathatggayta

SEQ ID NO: 4 - protein, 12 aa
KYPIPRERIMDY

SEQ ID NO: 5 - DNA (other nucleic acid), 32 bp; single-stranded, linear
  description: /desc = “PCR primer”
cccgaattctagaartayccnathccnagrca

SEQ ID NO: 6 - protein, 74 aa
TKTESSSRGPYHPSECCFTYTTYKIPRQRIMDYYETNSQCSKPGIVFITKRGHSVCTNPSDKWVQDYIKDMKEN

SEQ ID NO: 7 - DNA (cDNA), 123 bp; single-stranded, linear
tatgagaccagcagccagtgctccaagcccggaattgtcttcatcaccaaaaggggccattccgtctgtaccaaccccagtgacaagtgggtccaggactatatcaaggacatgaaggagaac

SEQ ID NO: 8 - protein, 47 aa
QRIMDYYETSSQCSKPGIVFITKRGHSVCTNPSDKWVQDYIKDMKEN

SEQ ID NO: 9 - DNA (genomic), 330 bp; single-stranded, linear
  CDS 11..151
gaattctagacagcggatcatggattactatgagaccagcagccagtgctccaagcccggaattgtcttcatcaccaaaaggggccattccgtctgtaccaaccccagtgacaagtgggtccaggactatatcaaggacatgaaggagaactgagtgacccagaaggggtggcgaaggcacagctcagagacataaagagaagatgccaaggccccctcctccacccacccctaactctcagccccagtcaccctcttggagcttccctgctttgaattaaagaccactcatgctcttcaaaaaaaaaaaaaaaatgagctctagaattc

SEQ ID NO: 10 - protein, 47 aa
QRIMDYYETNSQCSKPGIVFITKRGHSVCTNPSDKWVEDYIKDMKEN

SEQ ID NO: 11 - DNA (other nucleic acid), 35 bp; single-stranded, linear
  description: /desc = “PCR primer”
cctgaattctagagctcattttttttttttttttt